(19) United States
(12) Patent Application Publication
(10) Pub. No.: US 2005/0031645 A1
(43) Pub. Date: Feb. 10, 2005

(54) Vaccine compositions

(75) Inventors: John R. Murphy, Boston, MA (US); Edward O'Lear, Wolcott, CT (US); Robert J. Harrison, Medfield, MA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Advanced Microbial Solutions Corporation, Cambridge, MA (US)

(21) Appl. No.: 10/822,953

(22) Filed: Apr. 12, 2004

Related U.S. Application Data
(63) Continuation of application No. 09/868,753, filed on Jun. 21, 2001, now abandoned.
(60) Provisional application No. 60/161,292, filed on Oct. 25, 1999.
(60) Provisional application No. 60/161,193, filed on Oct. 22, 1999.

(51) Int. Cl. A61K 39/12; A61K 39/02; C12N 1/21

(52) U.S. Cl. 424/200.1; 435/252.3

(57) ABSTRACT

Disclosed are virulent or opportunistic prokaryotes in which metal ion-dependent gene regulation confers a growth or an infectious advantage. The prokaryote contains a DNA molecule containing a sequence encoding a dominant, metal ion-independent repressor protein or a partially metal-ion independent repressor protein. The prokaryotes are formulated into vaccine compositions and administered to a human or other animal to enhance protective immunity against infectious and diseases caused by prokaryotes in which metal ion-dependent gene regulation confers a growth or an infectious advantage.

### CROSS-REFERENCE TO RELATED APPLICATIONS

This application is a continuation of U.S. patent application Ser. No. 09/868,753 filed Jun. 21, 2001 which is a nationalization of PCT/US00/29231 filed Oct. 23, 2000, which claims the benefit of U.S. Provisional Application Nos. 60/161,292 filed Oct. 25, 1999 and 60/161,193 filed Oct. 22, 1999, the contents of all of which are hereby incorporated by reference.

### BACKGROUND OF THE INVENTION

Infectious disease remains the largest cause of mortality in the world. A significant proportion of infectious disease associated morbidity and mortality results from bacterial pathogens. One widely applied technique used in controlling the spread and severity of bacterial infection is vaccination. Several notable vaccine examples prevent a number of lethal microbial diseases, the DPT vaccine protects against diphtheria, pertusis and tetanus. The BCG or bacillus Calmette-Guerin vaccine is an example of an attenuated strain which is used worldwide to control the spread of tuberculosis (TB). A central issue in the development of safe and effective bacterial vaccines is the identification of protective antigens or attenuated strains of bacteria which can promote the development of an immune response in the host yet in vaccine composition fail to cause morbidity or martality in the host. A variety of methods can be employed to identify vaccine compositions including the production of attenuated or killed vaccine strains. Often these strains are in single genes which have been shown to play a role in virulence of the organism. Vaccine compositions can include a live or fixed bacterial preparation of the mutant strain, the fixed protein, a fusion protein made of the virulence gene product and a suitable carrier and more recently the DNA encoding the protein.

Attenuated strains offer the potential of presenting a nearly intact complement of pathogen associated antigens to immune system. Furthermore, these antigens are likely to be presented in a bacterial context that mirrors that seen during early stages of infection by a virulent strain. This is evidenced by the ongoing practice of using live attenuated strains in the extensive vaccination of both human and livestock. e.g., BCG for tuberculosis and strain 19 against bovine brucellosis and Sterne's spore vaccine against anthrax in cattle.

The use of live vaccines can present developmental obstacles including the retention of unacceptable levels of virulence, the risk of reversion to virulence during culture or in vivo and lack of efficacy. The ability to create more effective live or attenuated vaccines is in part dependent upon the ability to control and restrict the expression of virulence determinants so as to create vaccine strains that are protective and safe.

Bacteria respond to nutritional stress by the coordinated expression of different genes. This facilitates their survival in different environments. Among these differentially regulated genes are the genes responsible for the expression of virulence determinants. The selective expression of these genes in a sensitive or susceptible host allows for the establishment and maintenance of infection or disease. Virulence include genes which encode toxins, colonization factors and genes required for siderophores production or other factors that promote this process.

The expression of virulence genes in bacteria therefore enable the organism to invade, colonize and initiate an infection in humans and/or animals, however, these genes are not necessarily expressed constantly (constitutively). That is, the bacterium is not always orchestrating gene expression patterns to maximize “infectious” potential. In many circumstances, the expression of virulence genes is controlled by regulatory circuitry which include repressor proteins and a corresponding operon or operator. One class of repressors which is activated upon binding to or forming a complex with a transition metal ion such as iron, zinc or mangenese is thought to control the expression of a subset of genes in a number of Gram positive organisms. When such repressors are activated and associated with virulence gene expression in pathogens, they bind the operator sites thereby preventing production of virulence determinants.

Virulence determinants are most often expressed when the bacterial pathogen is exposed to environmental stress such nutritional restriction. An iron-poor environment is an example of such a condition. In many eucaryotes such an environment is the norm—insufficient iron is present to maintain the repressor in its active state. In the inactive form, the repressor cannot bind to target operators. As a result, virulence genes are de-repressed and the bacterium is able to initiate, establish, promote or maintain infection.

The expression of these virulence determinants is in many bacterial species co-regulated by metal ions. In most instances the metal co-factor that is involved in vivo is iron [but can include zinc, nickel, mangenese, cobalts]. In the presence of iron, the repressor is activated and virulence gene expression is halted.

This pattern of gene regulation is illustrated by the following example. The bacterium that causes diphtheria produces one of the most potent toxins known to man. The toxin is only produced under conditions of iron deprivation. In the presence of iron, the bacterial repressor (which in this species is known as diphtheria toxin repressor protein, abbreviated “DtxR”) binds iron and undergoes conformational changes that activate it and allow it to dimerize and bind a specific DNA sequence called the tox operator. The tox operator is a specific DNA sequence found upstream of the gene that produces the diphtheria toxin, thereby preventing its expression. Typically, during infection of a human host the diphtheria bacillus (or other pathogenic/opportunistic bacteria) grows in an environment that rapidly becomes restricted in several key nutrients. Paramount among these essential nutrients is iron, and when iron becomes limiting the diphtheria bacillus begins to produce the toxin. Moreover, the constellation of virulence genes that DtxR controls becomes de-repressed and the diphtheria bacillus becomes better adapted to cause an infection. In the case of diphtheria, the toxin kills host cells thereby releasing required nutrients including iron.

### SUMMARY OF THE INVENTION

A first aspect of the present invention is directed to a composition containing a virulent or opportunistic prokaryote in which metal ion-dependent gene regulation confers a growth or an infectious advantage. The prokaryote contains a recombinant DNA molecule comprising a promoter in operable association with a sequence encoding a dominant, metal ion-independent repressor protein or a partially metal ion independent repressor protein, and a carrier. In preferred embodiments, a promoter is constitutive in nature. In other preferred embodiments, the DNA molecule contains a sequence encoding a metal ion-independent DtxR protein or a partially metal ion-independent DtxR protein. In yet other preferred embodiments, the bacterium is a member of the genus Mycobacterium, Staphylococcus or Streptococcus.

The second aspect of the present invention is directed to a method of enhancing protective immunity against infection or disease caused by an opportunistic or virulent prokaryote pathogen in which metal or metal ion-dependent gene regulation confers a growth or an infectious advantage. The method entails administering the compositions to the animal. In preferred embodiments, the animal is a human. In other preferred embodiments, the compositions are administered prophylactically e.g. prior to the onset of the infection or disease. In yet other preferred embodiments, the prokaryote contained in the compositions is in a live or killed form.

A related aspect is directed to a method of attenuating or reducing the severity of an infection or a disease caused by an opportunistic or virulent prokaryotic pathogen in which metal or metal ion-dependent gene regulation confers a drug or an infectious advantage. The method also entails administering the compositions to the animal, preferably prior to the onset of the infection or disease condition.

Yet another aspect of the present invention is directed to isolated and purified DNA molecules consisting essentially of a sequence encoding a metal or metal ion-independent or a partially metal or metal ion-independent DtxR or homologue thereof. Preferred homologues are IdeR and SirR. In other preferred embodiments, the molecule is placed in an expression cassette or a vector (e.g., a plasmid) so as to be in operable association with a promoter element, especially a constitutive promoter. Vectors containing the DNA molecules and prokaryotes transformed with DNA molecules (including cultures thereof) are also provided.

A further aspect of the present invention is directed to a method for preparing the compositions. The method entails obtaining a DNA molecule encoding a metal ion-independent repressor protein or a partially metal ion-independent repressor protein. The wild-type protein, in its native state, is a metal ion-dependent gene regulator and confers upon a virulent or opportunistic prokaryote a growth or an infectious advantage. The DNA molecule is linked to a promoter, preferably a constitutive promoter, and then introduced into such a virulent or opportunist prokaryote. The DNA is expressed in the prokaryote and inhibits metal ion-dependent gene regulation.

The present invention entails the incorporation of an exogenous DNA encoding a dominant, metal ion (hereinafter “metal”) independent or partially metal ion independent mutant repressor into an otherwise virulent or opportunistic prokaryote in which metal ion-dependent gene regulation confers a growth or an infectious advantage. Such prokaryotes include gram-positive and gram-negative bacteria. Without intending to be bound by theory, Applicants believe that the dominance of the metal-independent mutant repressor subverts the normal patterns of gene regulation (under the control of the native, metal-dependent repressor), thereby creating a recombinant prokaryote that is attenuated or avirulent relative to the wild-type prokaryote. That is, the exogenous mutant repressor renders inoperable or significantly inoperable the normal metal-dependent genetic circuitry that occurs in vivo and causes in whole or in part the prokaryote carrying such a recombinant genetic compliment to become less infectious or non-pathogenic. This property renders the recombinant prokaryote suitable for use as an immunogen to be formulated into a vaccine.

### BRIEF DESCRIPTION OF THE DRAWINGS

FIG. 1 aligns the amino acid sequences of DtxR, IdeR and SirR.

FIG. 2 aligns amino acid sequences of various IdeR/SirR homologues found in various species of mycobacterium.

FIG. 3 aligns and compares the amino acid sequence of various homologues of various DtxR type repressors, including DtxR from Brevibacterium lactofermentum (Bl), DtxR from Corynebacterium diphtheriae (Cd), IdeR from Mycobacterium tuberculosis (Mt), M. leprae [P], M. smegmatis [P]; DesR from Streptomyces lividans (Sl), M.tuberculosis SirR, Staphylococcus aureus (Sa) SirR, S. epidermidis SirR, Enterococcus fecalis DtxR homologue [P], and the DtxR homologues from the Streptococcus gordonii, S. mutans, S. pneumoniae and S. pyogenes. The consensus amino acid sequences between these members of the DtxR family of iron-dependent repressors is indicated. *, metal ion coordination residues in the Primary site; #, metal ion coordination residues in the Ancillary site; @, the single amino acid residue that interacts with a base in the binding of DtxR dimers to the tox operator.

FIG. 4 is a Western blot of cell lysates incubated with polyclonal antibody against DtxR. Lane 1 shows purified DtxR (25.3 kDa). Lanes 3 and 5 show lysates from wild-type M.smegmatis and M.tuberculosis, respectively, expressing native IdeR (25.2 kDa.). Lane 4 shows lysate from the M.smegmatis heterodiploid harboring pNBV1/SAD expressing both DtxR(E175K) and IdeR. The molecular weight masses, determined by size standards, are shown on the left.

FIGS. 5A and B are bar graphs showing virulence comparison of wild-type M.tuberculosis and M.tuberculosis DtxR(E175K) mutant. FIG. 5A shows the log CFU of the homogenized spleens of mice sacrificed at 4 week intervals. FIG. 5B shows the log CFU of homogenized lungs at 4 week intervals. Each point represents the mean log CFU of 5-6 mice ±1 standard deviation (error bars). Asterisks denote statistically significant differences between groups at a given time point.

FIG. 6 is a photograph of a 10 week-old representative colony of wild-type M.tuberculosis (strain CDC1551) on 7H10 agar, and FIG. 6B is a photograph of a 10 week-old representative colony of M.tuberculosis DtxR(E175K) on 7H10 agar.

FIG. 7 is an alignment of the “iron box” consensus sequence, known DtxR binding sites, and putative M.tuberculosis DtxR/IdeR binding sites identified by an in silico genome search. The “consensus sequence” at the top of the figure represents the compilation of the 9 aligned sequences in the figure. The “published consensus” is drawn from the literature. Gene homologues of the downstream ORFs are shown on the right.

FIG. 8 shows autoradiographs of gel binding assays between DtxR and putative M.tuberculosis DtxR/IdeR binding sites, wherein 100 bp ³²P-end-labeled DNA fragments containing toxO (lanes 1 & 2), IB-1 (lanes 3 & 4), IB-2 (lanes 5 & 6), IB-3 (lanes 7 & 8), IB-4 (lanes 9 & 10), IB-5 (lanes 11 & 12) were separated in a non-denaturing 6% polyacrylamide gel. Odd numbered lanes contain DNA only (“unbound”), and even numbered lanes contain DNA pre-incubated with purified DtxR (“bound”).

FIGS. 9A and B are graphs showing that virulence of S. aureus is altered in a mouse skin lesion model following 8 days of in vivo incubation. 8.0 log CFU of the parent strain MA2181 [RN6390 carrying emtpy shuttle vector pSPT181] and 7.8 log CFU of the complemented MA2004 strain carrying DtxR E175K were injected sub-cutaneously on day 1. Abscess size[mm] was measured each day over 8 days and on the last day the abscess was removed and the number of CFU were determined. CFU [a.] and abscess size [b.] were compared between groups.

### DETAILED DESCRIPTION

To prepare the compositions of the present invention, a determination is made as to whether or not the species of interest regulates virulence determinant expression as a function of available metal ion concentrations. This can be done, for example, by screening protein from the bacteria of interest with an antibody for DtxR or other DtxR like proteins to ascertain if a homologous repressor exists in the species. This can also be accomplished using specialized techniques like gel mobility shift assays or the method disclosed in Sun, et al., PNAS 95:14985-14990 (1998), or more common gene expression monitoring methods such northern analysis and PCR. If the species of interest employs a DtxR type repressor than the expression of this repressor can be elucidated by one of the aforementioned methods and the techniques described here can be employed to build a recombinant attenuated strain for vaccine purposes.

Preferred prokaryotes are Gram positive bacterial species, and particularly those listed below. These species contain DtxR like metal dependent repressors. Specific examples include:

| | | |
|---|---|---|
| S. pneumoniae | S. agalactia | S. equisimillis |
| S. meningitis | S. bovis | S. anginosus |
| S. pyogenes | S. salivarius | S. sanguis |
| S. suis | S. mutans | Enterococcus faecalis |

Staphylococcus species

| | |
|---|---|
| S. aureus | S. epidermitis |

Mycobacteria species

| | | |
|---|---|---|
| M. tuberculosis | M. avium complex | M. kansasii |
| M. leprae | M. scrofulaceum | M. fortuitum |
| M. ulcerans | M. marinum | M. bovis |
| M. microtii | M. africanum | M. paratuberculosis |

Actinomyces species

| | | |
|---|---|---|
| A. pyogenes | A. israelii | A. bovis |
| A. viscosus | A. hordeovulneris | A. gerencseriae |
| A. naeslundii | A. odontolyticus | and others |

Listeria monocytogenes
Proprionibacterium acnes
Erysipelothrix rhusiopathiea

The repressor which typifies the class of genetic regulators in the above listed bacteria is the diphtheria toxin repressor DtxR in C. dihptheriae, the causitive agent of diphtheria. DtxR is a metal dependent repressor which under limiting concentrations of metal ions becomes inactivated permitting the derepression of a number of virulence genes including diphtheria toxin. This pattern of gene expression is common to both Gram-positive and Gram-negative bacteria. In Gram-positive bacteria, DtxR or DtxR homologues appear to be important metal dependent regulators whereas in Gram-negative bacteria, Fur is the significant metal dependent regulator. Some species of pathogen appear to contain both DtxR and Fur like metal dependent repressors. In each case, the presence of repressor bound metal ion is critical for appropriate activity of the repressor which coordinates the repression of gene expression in vivo.

The prokaryotes of the present invention having a dominant, metal ion independent or partially metal ion independent mutant of the repressor (such as the diphtheria toxin repressor gene DtxR or a DtxR-homolog e.g., IdeR, SirR) will render the pathogen unable to effectively establish a full infection. (Hill, et al., Infection and Immunity 66: 4123-4129 (1998); and Dussurget, ., Molecular Microbiology 22:536-544 (1996); and Pohl, et al., J. Molecular Biology 285:1145-1156 (1999)). Apart from the targeting vector sequences or plasmid DNA used to generate the attenuated strains and the mutant repressor gene, the recombinant prokaryote is in all other aspects identical genetically to the wild-type organism. The presence of the dominant iron-independent repressor results in a phenotypic change in the organisms' virulence.

In preferred embodiments, the exogenous DNA comprises a sequence encoding a dominant, metal-independent DtxR or a functional fragment, variant or homologue thereof (collectively referred to as “a DtxR protein”). DtxR is a metal iron-dpendent DNA-binding protein having a deduced molecular weight of 25,316 and which functions as a global regulatory element for a variety of genes on the C. diphtheriae chromosome. See Tao et al., Proc. Natl. Acad. Sci. USA 89:5897-5901 (1992); Schmitt et al., Infect. Immun. 59:1899-1904 (1994). For example, DtxR regulates the expression of the diphtheria toxin structural gene (tox) in a family of closely related Corynebacteriophages. The repressor has also been shown to regulate a number of other iron-dependent genes. The gene for DtxR and a number of DtxR homologues have been cloned and sequenced. A number of detailed structural and functional studies have be performed to analyse DtxR. See Boyd et al., Proc. Natl. Acad. Sci. USA 87:5968-5972 (1990); Schmitt et al., supra. DtxR is activated by divalent transition metal ions (e.g., iron). Once activated, it specifically binds the diphtheria tox operator and other related palindromic DNA targets. See Ding et al., Nature Struct. Biol. 3(4):382-387 (1996); Schiering et al. Proc. Natl. Acad. Sci. USA 92:9843-9850 (1995); White et al., Nature 394:502-506 (1998). DNA sequences encoding DtxR from various C. diphtheria strains are defined by accession numbers M80336, M80337, M80338, and M34239.

Functional fragments or variants of DtxR, when activated, retain their binding activity to the tox operator (or a functional fragment thereof) and/or the DtxR consensus binding sequence. DtxR fragments and variants can be identified by standard techniques such as mutagenesis. Tao et al., Proc. Natl. Acad. Sci. USA 90:8524-8528 (1993) identified important residues for DtxR function and analysis. Other variants are disclosed in Tao et al., Mol. Microb. 14(2):191-197 (1994). Tao discloses that some DtxR alleles have different amino acid sequences, e.g., the DtxR allele from strain 1030(−) of C. diphtheriae was found to carry six amino acid substitutions in the C-terminal region, none of which affected the iron-dependent regulatory activity of DtxR (1030) (Tao II). See also Boyd et al., J. Bacteriol. 174:1268-1272 (1992) and Schmitt et al., Infect. Immun. 59:3903-3908 (1991). Thus, DtxR fragments and variants may be mutagenized to an iron-independent phenotype.

Many other bacterial species employ regulatory circuits and repressor proteins that exhibit high degrees of sequence similarity to DtxR. (Posey, et al., Proc Natl Acad Sci 96:10887-10892 (1999), Que, et al., Molecular Microbiology :1454-1468 (2000), Kitten, et al., Infect and Immun 68:4441-4451 (2000), Manabe, Proc Natl Acad Sci 96:12844-12848 (1999). Thus, dominant, metal-independent DtxR homologues may also be employed in the methods of the present invention. Iron-dependent regulator (IdeR), isolated from Mycobacterium tuberculosis, has been found to share 60% amino acid homology with DtxR. See Schmitt et al., Infect Immun. 63(11):4284-4289 (1995). See also Doukhan et al., Gene 165(1):67-70 (1995), which reports and references DtxR homologs in Mycobacterium smegmatis and Mycobacterium leprae. DtxR homologues have been cloned in other gram-positive organisms including Brevibacterium lactofermentum and Streptomyces lividans. See Oguiza et al., J. Bacteriol. 177(2):465-467 (1995); Gunter et al., J. Bacteriol. 175:3295-3302 (1993); and Schmitt et al., Infect. Immun. 63:4284-4289 (1995). Staphylococcal iron regulated repressor (SirR), native to Staphylococcus epidermitis, is another known DtxR homologue. These proteins bear a common feature they share a remarkably high sequence similarity in the respective N-terminal 139 amino acid regions, especially those amino acids involved in DNA recognition and transition metal ion coordination.

A collection of accession numbers for sequences that are either homologous to DtxR or contain a consensus tox O/P is presented in Table 1. See http://www.ncbi.nlm.nih.gov/BLAST and http://www.ncbi.nlm.nih.gov/unfinishedgenomes.html. See also, Altschul, et al., J. Mol. Biol. 215:403-410 (1990); Gish, et al., Nature Genet. 3:266-272 (1993); Madden, et al., Meth. Enzymol. 266:131-141 (1996); Altschul, et al., Nucleic Acids Res. 25:3389-3402 (1997); and Zhang, et al., Genome Res. 7:649-656 (1997). This high degree of sequence similarity and homology is indicative of a widely conserved metal ion dependent regulatory pathway employing DtxR-family repressors. It is noteworthy that many important human and animal pathogens are present in this collection of bacteria. Dominant metal independent repressors can be generated and introduced inot a prokaryote. Such mutants alter virulent phenotype in vivo and may be used as vaccines.

TABLE 1
DtxR Homologs and Species with DtxR Binding Sites

| Pathogenic Human/Veterinary Applications | | Other | |
|---|---|---|---|
| CAA67572 | S. epidermitis | L35906 | C. glutamicum |
| Gi 1777937 | T. pallidum | Z50048 | S. pilosus |
| CAA15583 | M. tuberculosis | Z50049 | S. lividans |
| U14191 | M. tuberculosis | | |
| L78826 | M. leprae | | |
| M80336 | C. diphtheriae | | |
| M80337 | C. diphtheriae | | |
| M34239 | C. diphtheriae | | |
| M80338 | C. diphtheriae | | |
| AAD18491 | C. pneumoniae | | |
| Gi 3328463 | C. trachomatis | | |
| TIGR 1280 | S. aureus | | |
| OUACGT | S. pyogenes | | |
| Sanger 518 | B. bronchoseptica | | |
| Sanger 1765 | M. bovis | | |
| Sanger 520 | B. pertusis | | |
| WUGSC | K. pneumoniea | | |
| TIGR 1351 | E. faecalis | | |
| AE000783 | B. burgdorferi | | |
| TIGR 1313 | S. pneumoniea | | |
| Snager 632 | Y. pestis | | |
| AE001439 | H. pylori | | |
| TIGR 1752 | V. cholera | | |
| TIGR1O97 | C. tepidum | | |
| U14190 | M. smegmatis | | |
| Gi 2621260 | M. thermoautotrophicum | | |
| Gi 2622034 | M. thermoautotrophicum | | |
| M50379 | M. jannaschi | | |
| Q57988 | M. jannaschi | | |
| O33812 | S. xylosus | | |
| Gi 264870 | A fulgidus | | |
| Gi 2648555 | A fulgidu | | |
| Gi 2650396 | A fulgidus | | |
| Gi 2650706 | A fulgidus | | |
| BAA79503 | A. pernix | | |
| CAB49983.1 | P. abyssi | | |
| BAA30263 | P. horikoshi | | |
| AL109974 | S. coelicolor | | |
| L35906 | B. lactofermentum | | |
| Stanford 382 | S. meliloti | | |
| TIGR 76 | C. crescentus | | |
| TIGR 24 | S. putrificacieus | | |
| AE000657 | A. aeolius | | |
| TIGR 920 | T. ferrooxidans | | |

Preferred gram positive pathogenic bacteria include Mycobacterium bovis, Mycobacterium leprae, Mycobacterium paratuberculosis, Mycobacterium tuberculosis, Mycobacterium avium, Staphylococccus aureus, Staphylococcus epidermitis, Streptoccus mutans and Streptococcus pneumoniae. The preferred dominant DtxR (or homologue) repressors are metal independent and contain a single amino acid change to convert the native, metal-dependent repressor to metal independent regulation.

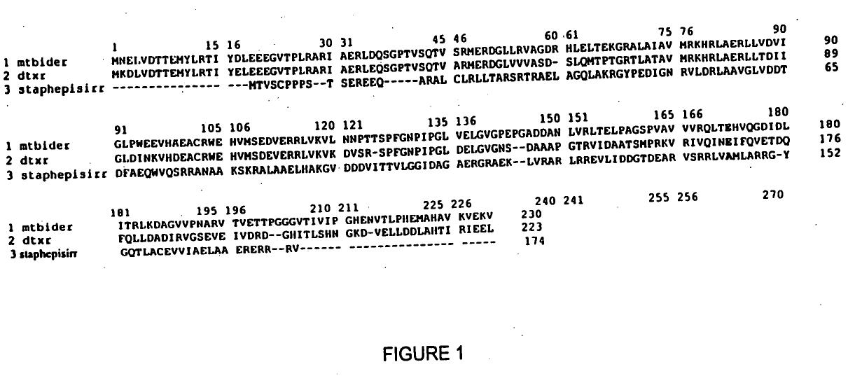

A preferred metal independent repressor is the mutant E175K DtxR. This same substitution can be made in DtxR fragments and variants. In addition, DtxR homologues that exist in other bacterial species may be mutated at the corresponding position. FIG. 1 displays the degree of amino acid homology between DtxR and the homologous proteins, IdeR from Mycobacterium tuberculosis and SirR from Staphylococcus epidermitis. As shown in FIG. 1, the single amino acid at residue E175K in DtxR, the glutamic acid is conserved in IdeR, hence a 177K mutation of IdeR would most likely have the analogous functional implications. Dussurget, et al., Molecular Microbiology 22:536-544 (1996) and Pohl et al.,J. Molecular Biology 285:1145-1156 (1999)).

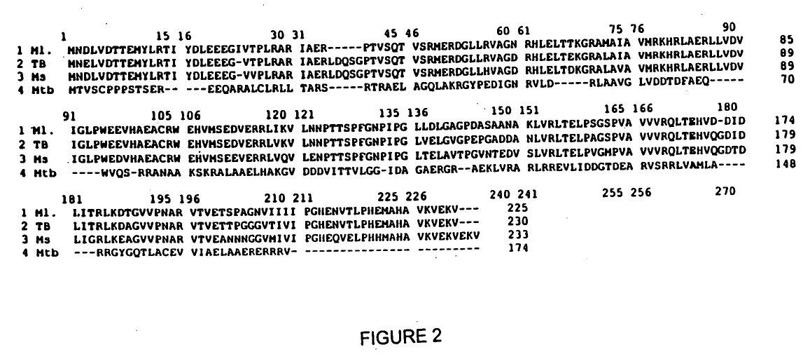

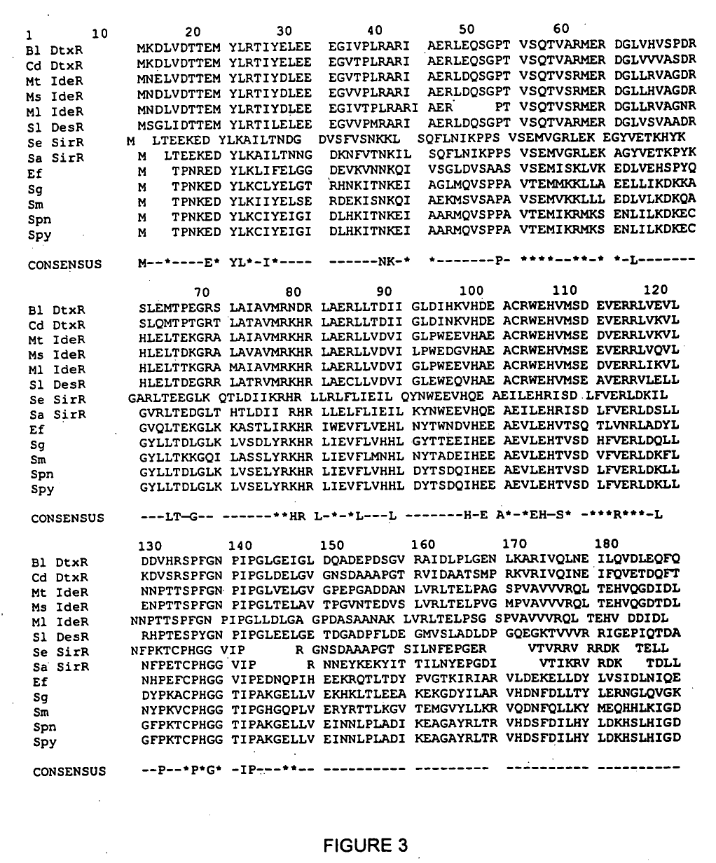

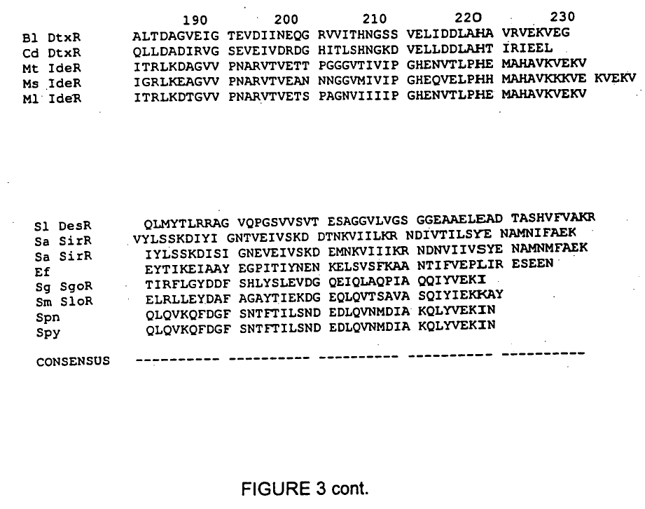

FIG. 2 presents a comparison of IdeR/SirR homologues found in other mycobacterium that cause significant disease including M. tuberculosis, M. smegmatis and M. leprae and a SirR clones from M. tuberculosis. Shown in bold is the conserved glutamic acid in the C-terminal region of these repressors that can potentially be mutated to yield an iron-independent version of each of these repressors. Thus, corresponding mutations would be expected to result in a metal-independent genotype. FIG. 3 illustrates the amino acid sequence homology of a number of homologous DtxR type repressors. These repressors differ slightly in their sequence length and the ClustalW program used to carry out a logical alignment adjusts for these differences. See NCBI [www.ncbi.nlm.nih.gov] web site and Baylor College of Medicine Search Launcher[http/.gc.bcm.tmc.edy] for details. Indicated by Bold ‘E’ is the highly conserved glutamic acid residue in the C-terminus of these repressors which is a possible target for generating metal ion independent versions of each of these represssors.

A combination of standard techniques may be used to make other dominant, metal-independent DtxR proteins, namely mutagenesis followed by tests to determine if a given mutant binds the corresponding operator. A number of theoretical mutations can be incorporated which will convert the repressor to become metal-independent. To generate repressor DNA clones with a random distribution of mutated bases, any of several saturation mutagenesis techniques can be utilized. DNA, plasmid preparation, and DNA sequence analysis are performed according to standard methods. The saturation of random mutations throughout the length of repressor DNA of interest introduces random changes in amino acid sequence throughout the encoded protein that potentially can confer a metal independent phenotype on the mutant repressor.

Several approaches can be pursued to generate effective vaccines relying upon DtxR. In preferred embodiments, DtxR homologues are identified in a species of interest. This can be achieved by performing PCR on genomic DNA from the species of interest using primer sets compatible to conserved domains within the DtxR family. Specific sequences may be composed of degenerate primers flanking the iron binding domain, or the helix-turn-helix domain. Cloning may also be performed from phage libraries using DtxR conserved regions as probes, utilizing the PSTD system or by in silico searches and de novo in vitro synthesis.

The DtxR has a domain structure which is composed of a helix-turn-helix domain, and N-terminal iron binding domain. There are a number of additional highly conserved sequences including a proline rich region which lies between the HLH and C-terminal domain. The polymerase chain reaction allows the amplification of any DNA target from a population of DNA molecules if compatible oligonucleotide primers can be identified. While a number of software programs are available to assist in the design of effective primers, optimal primer sets often require empirical determination.

Cloning of DtxR homologues from any given species can be achieved by mixing primers with genomic DNA isolated from the species of interest in the appropriate ratios in the presence of free oligonucleotides, optimized buffer conditions and the TAQ polymerase (or a suitable version of a thermostable polymerase) and cycling the reaction conditions with the aid of an automated thermocycler. The PCR reaction generates a series of products that represent target DNA sequences that are bounded by sequences homologous to the selected 5′ and 3′ primers. Thus, the selection of primers which lie within conserved regions of DtxR will likely bind under the appropriate conditions to homologous DNA that will likely represent a gene or domain similar to that of DtxR.

DtxR homologues may further be identified by screening genetic libraries of a given species created in E. coli. Pathogen libraries can then be screened by radio-labeled probes generated from dtxR clones, oligonucleotides from the dtxR sequence, or protein assays using antibodies directed against DtxR.

Sun, et al. have developed a screen can be used as a positive selection assay for DtxR homologues. See Sun, et al., PNAS 95:14985-14590 (1998). This screen requires only the generation of a gDNA library from the species of interest be transformed into the appropriate background host. Colonies which appear upon selection by chloramphenicol can only arise if a functional DtxR like protein is being expressed from the cloned GDNA fragment. Once a clone has been identified, sequencing and sequence analysis will reveal a gene which has at least partial sequence homology to DtxR and functional equivalence based on the PSDT screen developed by Sun et al.

Once identified, these clones become the substrates from which a vaccine strain is assembled. Two approaches are preferred. The first approach is a knock-in approach which is coupled with in vitro mutagenesis or PCR mutagenesis. The knock-in approach is focused on generating strains having dominant activated repressors. These strains contain a defective or altered copy of DtxR or a DtxR homologue that containing at least one but up to several mutations resulting in a repressor that recognizes and binds the toxAPO or suitable cognate binding sites in both the presence and absence of iron (or the appropriate metal ion). These metal-ion independent mutants are cloned into suitable vectors (e.g., targeting vectors having a selectable marker and restriction sites) to develop strains expressing dominant activated DtxR constitutively. These plasmids are used to generate knock-in vaccine strains, essentially altered forms of the wild-type virulent strain differing in only the presence of a dominant activated DtxR or DtxR-homologous repressor. The constructs can also be used in gene replacement strategies in which the mutant metal independent repressor gene replaces the endogenous wild-type gene. (Howard, et al., Gene 166:181-182 (1995), Jacobs, et al., Mthds in Enz 204:537-555 (1991), Rubin, et al., Proc Natl Acad Sci 1644-1650 (1999)), Caparon, et al., Mthds in Enz 204:556-586 (1991), Norgren, et al., Infect and Immun 57:3846-3850 (1989), Biswas, et al., J Bact. 175:3625-3635 (1993). Such a strain is effectively avirulent since its ability to up-regulate iron-dependent virulence genes has been crippled.

Metal ion-independent clones can be sequenced to identify the specific amino acid changes that confer the iron-independent phenotype. Again, a preferred mutant is characterized by a change from glutamic acid to lysine at position 175 in native DtxR. In general, however, multiple mutations are also embraced by the present invention. Multiple mutations are advantageous because they greatly decrease the likelihood of reversion to wild-type function. This likelihood becomes statistically more akin to that of a strain which has all of its iron regulated genes knocked out. Multiple mutations either clustered or distributed through out the repressor can result in the same phenotype. For example, a double mutant DtxR with the replacement of asparagine at position 130 with glycine and the glutamine at position 181 with arginine was also identified with a metal independent phenotype. In addition a mutant with an intermediate phenotype was also identified having a total of six mutations [valine 5 to isoleucine, aspartic acid 110 to glutamic acid, valine 112 to phenylalanine, isoleucine 153 to threonine, aspartic acid 197 to glutamic acid, threonine 220 to alanine]. These mutations produced a partially metal dependent phenotype. Thus, the present invention also embraces the use of partially metal ion-independent repressors. It is believed that expression of these repressors allows the partial colonization of the host which promotes the development of a robust protective immune response. The metal dependent phenotype can be readily revealed by linking a readily assayed reporter gene to the tox O/P in E. coli and assessing reporter function under metal and metal conditions as described by Sun, et al.

More generally, however, the DtxR mutants of the present invention are not limited to those characterized by single or multiple amino acid substitutions. Insertion and deletion mutants are also contemplated. These mutants may be identified using the instantly disclosed techniques as well.

Strains and putative mutants can be tested for metal independent phenotype by several approaches including the PSTD screen described in Sun et al. Additional methods of determining if conversion to iron-independent phenotype include computer modeling, structural analysis, dimerization analysis by gel electrophoresis, DNA binding by electrophoretic mobility shift assay, transcriptional profiling, tissue culture and in vivo virulence assays. Ultimately, in vivo screening will be required to determine if the iron-independent phenotype is stable in a host environment and to determine if virulence is attenuated. Attenuated virulence can be defined by biochemical, physiological and immunological markers but will minimally include an assessment of ED₅₀ or LD₅₀ in wild-type, knock-in, replacement and recombinant strains.

A second rational mutagenesis strategy can be utilized to generate metal-independent mutants. This strategy relies upon the conserved domain structure of the DtxR family of repressors. Several DtxR iron-independent mutants have been identified and published. These mutants define two classes of iron-independent mutations which are likely to alter iron-dependent regulation in DtxR homologues. Site directed mutagenesis of the analogous amino acid residues in DtxR homologues may have the same iron-independent phenotype. In addition, as the data from structural studies grow it will likely be possible to construct mutants that replace amino acids involved in the coordination of iron binding or dimerization that result in an iron-independent phenotype.

Mutagenesis is then carried out to generate a library of the desired mutants of this DtxR homologue of interest. Identification of iron-independent mutants from this population is achieved by using the PSTD system. Using the cloned and mutant homologue genes, one can select for the growth of colonies in the PSTD strains in the presence of dipyridyl (DP). Dipyridyl chelates iron from the media and therefore leads to the disassociation of DtxR homologues from the regulatory regions of the genetic elements in the PSTD screen. As a result, all iron-dependent repressors will not be able to survive chloramphenicol selection. In contrast, iron-independent mutants will grow.

Mutants of DtxR are generated in accordance with standard techniques. Polymerase chain reaction (PCR) mutagenesis of the dtxR gene is described in Vartanian et al. [Vartanian, J.-P., Henry, S., & Wain-Hobson, S. (1996) Hypermutagenic PCR involving all four transitions and a sizeable proportion of transversions. Nucleic Acid Res., 24, 2627-2631.]. Briefly, BglII-tagged primers 1515 (5′-ACCAGATCTGCCGAAAAACTTCGA-3′) and 1516 (5′-ACCAGATCTCCGCCTTTAGTATTTA-3′) were used to PCR amplify dtxR from plasmid pRDA which carries the wild-type dtxR operon. The products of the amplification were then digested with BglII and ligated either into BglII-linearized pSC6M1 and transformed into E. coli TOP10/λRS65T, or ligated into BamHI digested pBR322 and transformed into E. coli TOP10/λRS65T/pSC6. Iron-independent mutants of DtxR were then selected on LB agar plates supplemented with Cm and DP in accordance with the procedure described in Sun, et al.

Broadly speaking, these mutations should conserve the structural integrity and maintain the ability of the repressor to bind and repress gene expression through the consensus or near consensus tox P/O sites. Bacterial clones containing mutagenized dtxR can be analysed by DNA sequencing and used in functional biochemical assays such as electrophoeretic mobility shift assay, native gel analysis and gluteraldehyde crosslinking studies to reveal the activated state of the repressor in question under metal limiting conditions. This can be determined through gel shift analysis or by functional assays, but it is preferably made using the one-step method described by Frigg, et al. [Sun, L., vanderSpek, J. & Murphy, J. R. Proc. Natl. Acad. Sci. USA, 95:14985-14990 (1998)]. For gel shift analysis the native tox operator (i.e., 5′-ATAATTAGGATAGCTTTACCTAATTAT-3′) is a 27 base pair interrupted palindromic sequence upstream of the diphtheria tox structural gene can be used as a probe. This sequence features a 9-base pair inverted repeat sequence that is separated by 9 base pairs. See Kaczorek et al., Science 221:855-858 (1983); Greenfield et al., Proc. Natl. Acad. Sci. USA 80:6853-6857 (1983); Ratti et al., Nucleic Acids Res. 11:6589-6595 (1983); and Fourel et al., Infect. Immunol. 57:3221-3225 (1989). It overlaps both the −10 region of the tox promoter and the transcriptional start sites at −45, −40 and −39 upstream of the diphtheria toxin structural gene. See Boyd et al., J. Bacteriol. 170:5940-5952 (1988). The minimal essential DNA target site, i.e., 5′-GTAGGTTAGGCTAACCTAT-3′, is a 19 base pair sequence that forms a perfect palindrome around a central C or G,and is described in Tao and Murphy, Proc. Natl. Acad. Sci. USA 91:9646-9650 (1994). Additional probes are variants of ToxO based on the DtxR consensus-binding sequence (5′-ANANTTAGGNTAGNCTANNCTNNNN-3′). Variants are defined by the following sequence: 5′-TWAGGTTAGSCTAACCTWA-3′. Thus the function of the repressor and mutant can be defined by recognition and binding or regulation of gene expression via the sequences or variants described above.

Once a dominant, metal-independent DtxR DNA clone is identified, it can be produced and manipulated in accordance with techniques known in the art. For example, they may be generated using standard chemical synthesis techniques. See, e.g., Merrifield, Science 233:341-347 (1986) and Atherton et al., Solid Phase Synthesis, A Practical Approach, IRL Press, Oxford (1989). Preferably, they are obtained by recombinant techniques. Standard recombinant procedures are described in Sambrook et al., 1989, Molecular Cloning, A Laboratory Manual, Second ed., Cold Spring Harbor, N.Y., and Ausubel et al., (eds.) Current Protocols in Molecular Biology, Green/Wiley, N.Y. (1987 and periodic supplements). The appropriate sequences can be obtained from either genomic or cDNA libraries using standard techniques. DNA constructs encoding the DNA gene segments may also be prepared synthetically by established methods, e.g., in an automatic DNA synthesizer, and then purified, annealed, ligated and cloned into suitable vectors. Atherton et al., supra. Polymerase chain reaction (PCR) techniques can also be used. See e.g., PCR Protocols: A Guide to Methods and Applications, 1990, Innis et al. (ed.), Academic Press, New York.

The DNA encoding the mutant metal-independent repressor is operably linked to a promoter element. Preferred promoters include the endogenous DtxR promoter or the promoter of the DtxR homologue in question or any suitable constitutive promoter functional in the species of interest. The constructs may further contain an associated selectable marker gene to follow maintenance of the mutant construct. For example, in Mycobacterial species, a hygromycin resistance gene in an E. coli shuttle vector provides great utility for cloning and expression of a mutant IdeR or another dominant metal ion independent repressor [DtxR E175K]. See, Bishai et al., Gene 166:181-182 (1995). In other embodiments, the vectors also contain a copy of a gene lethal to the bacterium under the control of the metal dependent regulator. Examples of such genes include antibiotic genes, restriction enzymes, proteolytic enzymes, lethal phage genes or any gene whose product once expressed would kill the bacterium. The presence of this “suicide cassette” further ensures that vaccines containing the prokaryote in live form do not revert to any significant degree and cause disease in vivo.

Suitable cloning and expression vectors are readily available from a number of sources. In preferred embodiments, the construct is introduced into the prokaryote by way of a vector, in which case the construct may be formed prior to or upon introduction of the DNA into the vector. A vector for a given species must contain an origin of replication, a selectable marker and a functional promoter by which the mutant metal-ion independent repressor can be expressed in the strain of interest. (Howard et al., Gene 166:181-182 (1995), Jacobs et al., Mthds in Enz 204:537-555 (1991), Rubin et al., Proc Natl Acad Sci 1644-1650 (1999)), Caparon et al., Mthds in Enz 204:556-586 (1991), Norgren, et al., Infect and Immun 57:3846-3850 (1989), Biswas et al., J Bact. 175:3625-3635 (1993).

The vectors are introduced into the cells in accordance with standard techniques such as transformation, co-transformation, direct transfection (e.g., mediated by calcium phosphate or DEAE-dextran) biolistics and electroporation. The recombinant cells are then cultured via standard techniques. Conditions may vary depending upon the prokaryote (e.g., bacterial species) being used. In general, culturing is continued from about 24 to 48 hours at a temperature between about 30 and about 39° C., preferably 37° C. The recombinant cells are cultured in an appropriate complete medium containing a selectable marker to assure a pure population. The strains can also be counter engineered as described below so that if significant levels of the metal independent repressor is not produced, a suicide gene will be de-repressed resulting in the death of the vaccine strain in situ. Iron is an essential element for both the bacterial pathogen and its animal host; thus, successful competition for this element is an essential component of the infectious process. The concentration of free iron in the mammalian host available to an invading bacterial pathogen is also extremely limited. As a result, the expression of virulence determinants (e.g., colonization factors, siderophores, hemolysins and toxins) by bacterial pathogens is regulated by iron. Accordingly, the vaccine cultures must be propageted in a complete medium containing iron and other divalent metal cations to facilitate their proliferation.

The vaccines of the present invention may be used in a wide variety of vertebrates, particularly man and domestic animals such as bovine, ovine, porcine, equine, caprine, domestic fowl, Leporidate, or other animals that may be held in captivity or may be a vector for a disease affecting a domestic vertebrate.

Pathogens of interest include any specie of microorganism which causes disease and relies entirely or partially upon a repressor mediated regulation of metal-dependent virulence. The present invention relates to methods of vaccinating a host with live recombinant bacteria to elicit protective immunity in the host. The recombinant vaccine can be used to produce humoral antibody immunity, cellular immunity (including helper and cytotoxic immunity) and/or mucosal or secretory immunity.

The manner of application of a vaccine strain may be varied widely, any of the conventional methods for administering an attenuated vaccine being applicable. These include aerosol applications, oral applications, in drinking water, on a solid physiologically acceptable base, or in a physiologically acceptable dispersion, parenterally (e.g., subcutaneously, intramuscularly, intravascularly or intraperitoneally), by injection, by in ovo inoculation or the like. The dosage of the vaccine (e.g., number of prokaryotic cells, number of administrations, period of administration, etc.) will vary according to the vaccine strain used and the species, age, and size of host to be protected. Persons skilled in the art will be able to determine the doseage to be administered so as to provide a sufficient immune response. The recombinant prokaryotes in the composition may be “live” or in “killed” form, as these terms are commonly used in the vaccine art.

The formulation of vaccine strain compositions may also vary widely. Pharmaceutically acceptable vehicles such as water are expected to be useful for oral administration. Other such vehicles including normal saline may be used for parenteral, cloacal or other routes of administration. The vaccine compositions may also be admixed with food for some applications.

The following example is not intended to limit the scope of the invention in any manner.

### EXAMPLE 1

Attenuation of Virulence in Mycobacterium tuberculosis Expressing a Constitutively Active Iron Repressor

This example describes the construction of a candidate strain which is severely attenuated with respect to the parent wild-type strain yet which persists long enough to allow the host to mount an immune response.

Summary

With over one-third of the world's population latently infected with Mycobacterium tuberculosis, the global burden of tuberculosis is staggering. The emergence of multi-drug resistant strains and the increased susceptibility of the HIV-infected further highlights the need for elucidation of the molecular pathogenesis of M.tuberculosis and its virulence genes.

Iron plays a critical role in the regulation of virulence of many bacterial pathogens (1). In tuberculosis, there is indirect clinical and in vitro evidence that iron regulation is important to the virulence of this microbial pathogen (2-5). Iron is an essential nutrient for the survival of most organisms and has played a central role in the virulence of many infectious disease pathogens. Mycobacterial IdeR is an iron-dependent repressor that shows 80% identity in the functional domains with its corynebacterial homologue, DtxR. In a novel approach to attenuation, Mycobacterium tuberculosis has been transformed with a vector expressing an iron-independent, positive dominant, corynebacterial DtxR hyperrepressor, DtxR(E175K). Western blots of whole cell lysates of M. tuberculosis expressing the dtxR(E175K) gene revealed the stable expression of the mutant protein in mycobacteria. BALB/c mice were infected by tail vein injection with 2×10⁵ organisms of wild-type or M.tuberculosis transformed with the dtxR mutant. At 16 weeks, there was a 1.2 log reduction in bacterial survivors in both spleen (p=0.0002) and lungs (p=0.006) with M. tuberculosis DtxR(E175K). A phenotypic difference in colonial morphology between the two strains was also noted. A computerized search of the M. tuberculosis genome for the palindromic consensus sequence to which DtxR and IdeR bind, revealed six putative “iron boxes” within 200 base pairs of an open reading frame. Using a gel shift assay, it was shown that purified DtxR binds to the operator region of five of these. Attenuation of Mycobacterium tuberculosis can be achieved by the insertion of a plasmid containing a constitutively active, iron-insensitive repressor, DtxR(E175K), which is a homologue of IdeR. The results demonstrate that IdeR controls genes essential for virulence in M.tuberculosis.

In a phylogenetically related organism, Corynebacterium diphtheriae, iron depletion results in the derepression of virulence genes such as the diphtheria toxin (tox) gene by DtxR (diphtheria toxin repressor). The corynebacterial DtxR has a homologue in M. tuberculosis, IdeR (iron-dependent repressor). In the amino terminal 140 amino acids that contain the Fe²⁺ and DNA-binding domains of DtxR, IdeR shares 80% identity with DtxR (6). In 1995, ideR was first described by Doukhan et al. in conjunction with the sigA sigB cluster of genes (7). Subsequently, the ability of mycobacterial IdeR to bind to the corynebacterial tox operator region in a metal ion-dependent manner was demonstrated by gel shift assay (8). Mutation of ideR in M.smegmatis resulted in derepressed siderophore production in high iron conditions (9). These findings parallel those described in corynebacterial dtxR and suggest that the homology between these two genes may allow for cross-genus functional complementation.

Using a positive genetic selection system to clone dtxR alleles, Sun et al. isolated and characterized a series of DtxR mutants created by PCR mutagenesis (10). One of the mutants which bound to the tox operator (toxO) and constitutively repressed reporter gene expression in an iron-independent manner was characterized and found to have a single amino acid substitution of lysine for glutamic acid at position 175 (DtxR(E175K)). In merodiploid strains harboring both wild-type dtxR and mutant dtxR(E175K) genes, Sun et al. found the mutant to be dominant over the wild-type allele.

Methods

Strains, Plasmids, and Cultures The bacterial strains and plasmids used in this study are listed in Table 2, set forth at the conclusion of this example (10-14). Escherichia coli cultures were grown in Luria broth or Luria agar supplemented with ampicillin (100 g/ml) or hygromycin (200 g/ml). M. tuberculosis CDC 1551 and M. smegmatis cultures were grown in standard Middlebrook 7H9 broth (Difco), supplemented with albumin dextrose complex (ADC), 0.1% glycerol, and 0.05% Tween 80 at 37° C. in roller bottles (15).

Construction of dtxR (E175K) Shuttle Vector Plasmid A 1.5 kb BamHI-HindIII fragment of DNA from pSDM2 was cloned into pNBV1. The resulting recombinant plasmid, pNBV1/SAD was cloned in E.coli DH5 and purified using the Qiagen system (Qiagen, Chatsworth, Calif.) (16). Purified plasmids were then electroporated into M. tuberculosis CDC1551 by standard protocols (15).

Western Blot Analysis Recombinant E.coli and mycobacteria were lysed in 3M urea, 0.5% Triton X-100, 3.25M DTT, 2% Pharmalyte (Pharmacia Biotech, Piscataway, N.J.), PMSF (100 g/ml) and leupeptin (2 g/ml). Using 0.1 mm glass beads, the samples were homogenized twice in a Mini-bead-beater (Biospec Products, Bartlesville, Okla.) at maximum speed for 1 minute. Samples were centrifuged to remove cellular debris and unlysed cells. After separation by 12% SDS polyacrylamide gel electrophoresis, proteins were transferred to nitrocellulose membranes (Hybond, Amersham, Buckinghamshire, UK) by semi-dry technique (Transblot S.Dak., Hercules, Calif.) and blocked with 5% non-fat milk in PBS with 0.1% Tween 20 (PBS-T) for 1 hour. Membranes were then incubated overnight in PBS-T with rabbit anti-DtxR polyclonal antibodies at the appropriate concentration at 4° C. (17). After washing, membranes were incubated with horseradish peroxidase-conjugated anti-rabbit antibody diluted in PBS-T for 2 hours. The Supersignal Chemiluminescent Substrate (Pierce, Rockford, Ill.) was used for autoradiograph development.

Murine Tuberculosis Infection Model 6-8 week-old BALB/c mice were infected by tail vein injection with 2×10⁵ organisms of wild-type or M.tuberculosis DtxR(E175K). Bacterial infection was monitored over a 119-day period. Colony forming units (CFU) in spleen and lungs were assessed at 4 week intervals by serial dilutions of organ homogenates plated on 7H10 Middlebrook agar containing cycloheximide (50 g/ml), carbenicillin (50 g/ml), trimethoprim (20 g/ml), and polymyxin (200 units/ml) (18).

DNA Gel Shift Binding Assay The DNA migration retardation assay was performed as previously described (19). Purified DtxR protein was isolated by methods as described (20). Radiolabeled DNA iron box fragments were generated by PCR using 100 ng of ³²P-end-labeled primer mixed with 150 ng of unlabeled primer and template DNA from gel-purified 100 bp cold fragments containing the iron box of interest. Binding reactions were carried out in 10mM Tris-OAc (pH7.4), 1 mM EDTA, 50 mM KCl, 1 mM DTT, 5% glycerol, 50 g/ml calf thymus DNA. Binding reactions were equilibrated for 30 minutes and then loaded onto a non-denaturing 6% acrylamide gel (21).

Results

Expression of the Corynebacterial dtxR Gene in Mycobacteria

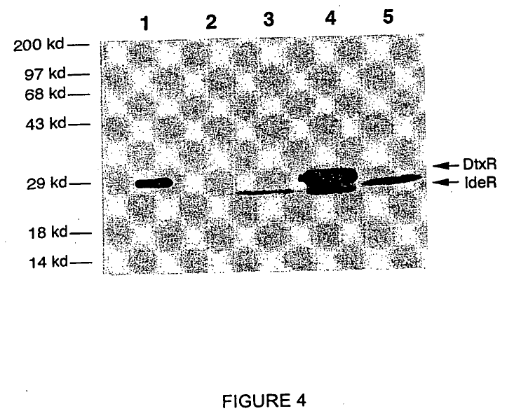

The 1.5 kb corynebacterial DNA fragment cloned in pNBV1/SAD contained 500 bp of 5′ non-coding sequences as well as the entire dtxR(E175K) open-reading frame. To determine if the corynebacterial mutant dtxR(E175K) gene was expressed in mycobacteria, we transformed M. smegmatis, a fast growing strain of mycobacteria, with pNBV1/SAD. Whole cell lysates prepared from M. smegmatis cultures were separated by 12% SDS PAGE. FIG. 4 shows a Western blot developed with polyclonal anti-DtxR antibodies. As illustrated, these antibodies recognize both DtxR and IdeR because of their significant antigenic similarity. Although the deduced molecular mass of IdeR (25.2 kDa) differs by only 0.1 kDa from DtxR (25.3 kDa) we have repeatedly observed anomalous accelerated migration of IdeR in our SDS-PAGE gels in which it runs at 23 kDa in spite of its mass of 25 kDa. This phenomenon has also been noted by Schmitt et al. (8) In preparations from M.smegmatis harboring pNBV1/SAD (FIG. 4, lane 4), two distinct bands appear. Because dtxR(E175K) is expressed from a multicopy plasmid, significantly more DtxR(E175K) protein is made than the chromosomally expressed IdeR. Similar results in M.tuberculosis transformed with pNBV1/SAD were also found (results not shown). The in vitro growth rate of wild-type M. tuberculosis was indistinguishable from that of M. tuberculosis DtxR(E175K) by the BACTEC radiometric growth monitoring system.

Attenuation of Virulence in M. tuberculosis Expressing the Constitutively Active DtxR Hyperrepressor After confirming that the corynebacterial mutant dtxR was expressed in transformed mycobacteria, we turned to an in vivo animal model to test the effect of the hyperrepressor on virulence. Forty-eight BALB/c mice were inoculated with 2×10⁵ CFU of CDC1551 M. tuberculosis or M. tuberculosis DtxR(E175K) by tail-vein injection. Both animal weights and the tissue burden of surviving bacteria were monitored over time. Mice infected with wild-type M.tuberculosis began to lose weight beginning at 13 weeks while the M. tuberculosis DtxR(E175K)-infected animals initially gained weight, then maintained stable weights for the duration of the experiment. At 17 weeks, there was a statistically significant difference of 1.7 gms (p=0.006 by two-tailed t-test) between the wild-type and DtxR(E175K) groups.

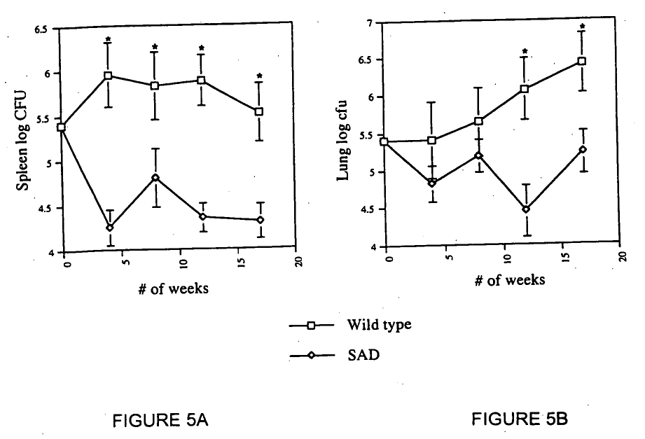

FIGS. 5A and 5B show the survival of the two M. tuberculosis strains in lungs and spleens of mice over time. At 17 weeks, there was a 1.2 log attenuation in virulence of the DtxR(E175K) expressing strain compared with wild-type which was statistically significant in both spleen (p=0.0002) and lungs (p=0.006). Analysis of the colonies from the mouse tissues at 12 weeks showed that 99% of the colonies were hygromycin-resistant indicating maintenance of the pNBV1/SAD plasmid. Histopathologic inspection of spleen and lungs of wild-type and DtxR(E175K) expressing strains corroborated our CFU data with fewer visible acid fast bacilli at 17 weeks in histologic sections of mouse organs from animals infected with the M. tuberculosis DtxR(E175K) than with the wild-type.

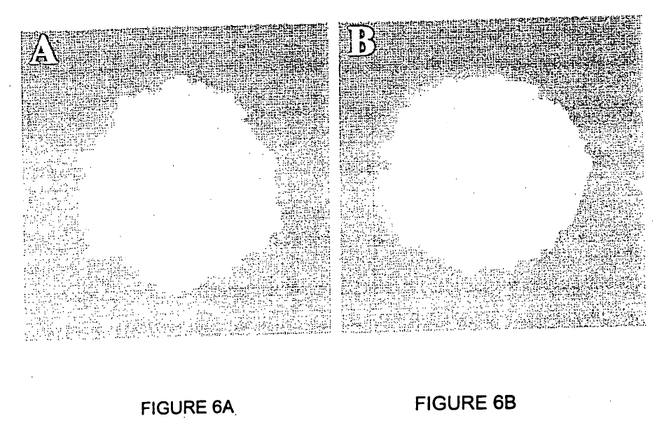

Differences in Colonial Morphology Between Strains Colonies of M. tuberculosis DtxR(E175K) grown from frozen stocks on 7H10 Middlebrook agar showed no difference in growth rate in vitro as compared to wild-type CDC1551, but were noted to have a distinct colonial morphology (see FIGS. 6A and 6B). The recombinant strain colonies were rougher and drier-appearing and were more raised and wrinkled than wild-type colonies. In addition, yellow pigmentation was also noted in the DtxR(E175K) expressor. Both strains exhibited a spreading phenotype and were crenelated at the periphery.

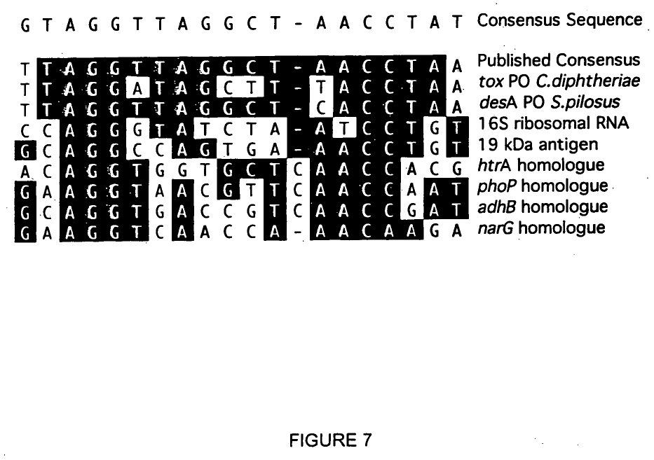

Identification of Iron Boxes An imperfect palindromic consensus sequence of the “iron box” for DtxR/IdeR has been established by in vivo and in vitro methods (8, 22, 23). This consensus sequence is listed at the top of FIG. 7. To identify genes that may be regulated by IdeR, we searched the M. tuberculosis genome for iron boxes that were in untranslated regions within 200 bp of an open reading frame. We chose two half-site sequences with allowance for a variable number of intervening base pairs for our search. In the 4.41 MB of the M. tuberculosis genome (24), 58 sequences with acceptable homology to the consensus sequence were identified. Six of these were in untranslated regions and had corresponding downstream open reading frames.

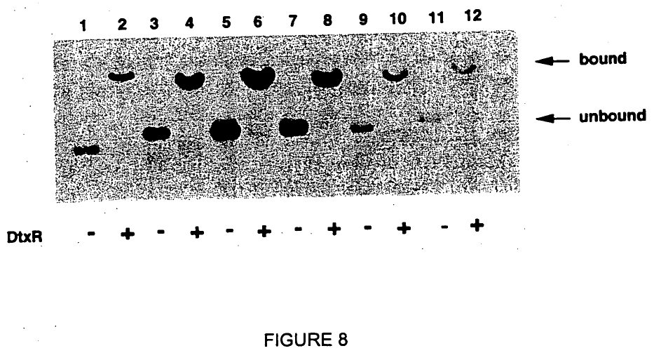

A DNA gel binding assay was used to assess the ability of DtxR to bind to these putative iron-regulated operator regions drawn from the M.tuberculosis genome. FIG. 8 shows the results of gel binding assays using ³²P-end-labeled 100 bp DNA fragments containing five of the putative iron boxes (IB1-5). Binding of DtxR to the tox operator could be abolished with the addition of unlabeled tox DNA, but not with nonspecific DNA. All five of these putative iron boxes were bound by DtxR to a similar degree as that observed with the tox operator. The iron box upstream of the narG homologue, IB6, did not bind to DtxR (data not shown).

Table 3 identifies the open reading frames (ORF) downstream of these six iron boxes. BLAST searches reveal that these genes encode a PhoP homologue (a transmembrane sensor of a two-component sensor-regulator pair), a homologue of the HtrA serine protease, 16S ribosomal RNA, an alcohol dehydrogenase AdhB, and a homologue of the M. tuberculosis 19kDa antigen (a protein shown to be involved in the human immune response to tuberculosis) (25). IB6, which was not shifted by DtxR in vitro, appears upstream of a nitrate reductase subunit gene, narG.

The concentration of free ferrous iron (Fe²⁺) is extremely limited in vivo. For this reason, many pathogenic prokaryotes such as Vibrio cholerae, E.coli, Neisseria gonorrheae, and Corynebacterium diphtheriae co-regulate virulence gene expression with iron sensing and scavenging systems (26-28). In C.diphtheriae, one such mechanism of iron regulation relies on a repressor, DtxR, which binds to a specific palindromic sequence in the operator regions of the genes that it controls (29). In low iron states, the metal-ion triggered conformational change that allows it to bind to the DNA is disrupted, the repressor loses affinity for the operator site, and gene expression occurs. Recently, a positive dominant DtxR(E175K) mutant unresponsive to iron was generated by random PCR mutagenesis using a genetic selection system (10).

Significant amino acid identity between corynebacterial DtxR and mycobacterial IdeR has been described. In the amino terminal 140 amino acids there is a DNA binding helix-turn-helix motif, a primary metal ion binding site and a protein-protein interaction domain. Corynebacterial DtxR and mycobacterial IdeR share 80% amino acid identity in this portion of both proteins. Evidence of functional homology between IdeR and DtxR has been shown previously by Schmitt et al. (8).

The results show that the positive dominant DtxR(E175K) iron-independent repressor is expressed in the phylogenetically related mycobacteria. Furthermore, we have shown that it is dominant and constitutively attenuates M. tuberculosis in a murine model of infection. Rational attenuation of M. tuberculosis provides the opportunity to define specific virulence factors of the organism and the development of live vaccines superior to BCG. However, gene replacement has proven difficult in M. tuberulcosis due to high rates of illegitimate recombination. Addition of a dominant mutant gene is technically simpler than gene replacement in M. tuberculosis and permits comparison of a defined merodiploid strain with an isogenic wild-type strain. There are reports of E. coli genes introduced into other bacteria to regulate the expression of endogenous genes. In a paper by de Henestrosa et al., a mutant E. coli recA gene produced aberrancies in SOS gene induction when expressed in heterologous gram-negative systems (30). Although a M. tuberculosis strain containing empty plasmid was not compared with wild-type M. tuberculosis, the in vitro Bactec comparison showed no differences in the rate of growth of the mutant strain as compared to wild-type. In addition, studies of deletion mutants have shown that plasmid complementation fully restores virulence suggesting that there is little cost to the organism to maintain the plasmid (31, 32). This strain is the first example of the use of a dominant positive gene from another pathogenic prokaryote to attenuate the virulence of M. tuberculosis.

Animal models have shown that inactivation and clearance of virulent M. tuberculosis in liver and spleen are effectively accomplished, but that the same cell-mediated immune mechanisms appear relatively ineffective in lungs. These data point to a difference in the intracellular microenvironment of the lung granuloma (33). It has been postulated from BCG and H37Ra data that avirulent or attenuated strains lack the genes required for effective growth within lung phagocytes (34). The data suggest that IdeR may regulate genes important for M. tuberculosis survival late in lung infection as attenuation seems to increase dramatically at 12 weeks after infection. This may correlate with the onset of granuloma formation in mouse lungs and the need for M. tuberculosis to scavenge iron from extracellular rather than intracellular sources (35).

The ideR gene has been found in M. tuberculosis, M. bovis, and M. smegmatis. In M. smegmatis, an ideR mutant showed defective regulation of siderophore biosynthesis. (9) Potential IdeR binding sites upstream of exochelin biosynthesis genes such as fbA have recently been confirmed (36, 37). In addition, several IdeR recognition sequences have been identified using computer searches of the M. tuberculosis genome (38). We have similarly identified 6 potential IdeR-binding sites in M. tuberculosis, 5 of which demonstrated significant binding with DtxR in a gel shift assay. We postulate that the sixth sequence was unable to bind in our in vitro assay because of incorrect spacing between the two relatively well-conserved half-sites. We used DtxR rather than IdeR in this gel-shift assay because we specifically sought to identify genes responsible for the attenuated phenotype of M. tuberculosis DtxR(E175K). The predicted ORF downstream of IB-1 encodes a homologue of phoP, a phosphotransfer response regulator. A number of two-component pairs have been shown to regulate virulence pathways in bacterial pathogens. These include BvgA/BvgS in Bordatella pertussis, VanR/VanS in Enterococcus faecium, PhoP-PhoQ in Salmonella typhimurium, and OmpT/EnvZ in Shigella flexneri (39, 40). In M. tuberculosis, a two-component pair, mtrA-mtrB, has been previously described and appears to play an intracellular role as expression of mtrA increases upon entry into macrophages (41). Furthermore, phoP- mutants in Salmonella are unable to synthesize many of the proteins expressed on interaction with macrophages (42). Downstream of IB-2 is adhB, an alcohol dehydrogenase. In Salmonella typhimurium, it has been postulated that alcohol dehydrogenase genes such as eutG may confer a protective role from reactive aldehyde intermediates associated with inflammatory cell activation (43).

IB-3 lies upstream of an ORF homologous to a HtrA-like serine protease which, in E.coli are thought to be required for growth of the organism at high temperature, and may play a role in degrading abnormal proteins within the periplasm (44, 45). It is a known virulence factor in several organisms including Salmonella typhimurium, Yersinia enterocolitica, Brucella abortus, and Brucella melitensis (46-49). In an animal protection model, a Salmonella typhimurium htrA mutant is attenuated and a safe and immunogenic live vaccine strain in mice (50). Both Mycobacterium avium subsp. paratuberculosis and M. tuberculosis have putative serine proteases with significant homology to HtrA (24, 51).

IB-4 lies upstream of rrnA, a 16S rRNA gene which has been shown to be part of a group of rDNA operons in both slow and fast-growing mycobacteria with hypervariable multiple promoter regions (HMPR). The M.tuberculosis rrnA operon has 2 promoters one of which is conditionally induced suggesting complex regulation of this esential gene (52).

Our results indicate that a dominant positive corynebacterial dtxR allele attenuates the virulence of M.tuberculosis in a murine model. These data implicate the M.tuberculosis IdeR repressor as a regulator of genes essential for full virulence.

TABLE 2

| Strain/Plasmid | Genotype/Description | Source/Reference |
|---|---|---|
| Plasmids | | |
| PNBV1 | E. coli-mycobacterial shuttle vector | (12) |
| Ap^{R}, Hy^{R} | | |
| pSDM2 | pSC101-derivative containing the | (10) |
| | dtxR(E175 K) gene, Km^{R} | |
| pNBV1/SAD | pNBV1 containing the dtxR(E175 K) | this paper |
| gene, Ap^{R}, | | |
| Hy^{R} | | |
| Strains | | |
| E. coli DH5 | F⁻ recA1, hsdR17, thi-1, gyrA96, | (14) |
| | supE44, endA1, relA1, recA1, | |
| | deoR, (lacZYA-argF) U169 | |
| | (80 lacZ M15) | |
| M. smegmatis | transformable variant of mc²6 | (13) |
| mc²6 1-2C | | |
| M. tuberculosis | virulent recent clinical isolate | (11) |
| CDC1551 | | |
| M. tuberculosis | pNBV1/SAD | this paper |
| DtxR(E175K) | | |

Hy hygromycin, Ap ampicillin, Km kanamycin

TABLE 3
Iron Boxes (IB)

| | Downstream | Accession | |
|---|---|---|---|
| Name | ORF | Number | Description |
| IB-1 | PhoP | Rv0761c | Two-component |
| | homologue | | phosphotransferase |
| | | | regulatory protein |
| IB-2 | AdhB | Rv0757 | Alcohol dehydrogenase |
| IB-3 | HtrA | Rv0983 | Serine protease, HtrA-antigen |
| | homologue | | family |
| IB-4 | RrnA | MTB00368 | 16S ribosomal RNA protein |
| IB-5 | Hypothetical | Rv3764c | Predicted ORF with 26% similarity |
| | protein | | to M. tuberculosis 19 kDa antigen |
| | | | beginning at base 4,210,314 |

### EXAMPLE 2

Attenuation of Staphylococcus Infection in a Murine Model

In Example 1 Applicants demonstrated the ability of an in vitro constructed and tested metal ion-independent mutant gene of a normally metal ion dependent transcriptional repressor to attenuate virulence in a pathogenic bacterium of a different species. The metal ion dependent repressor was from C. diphtheriae and the pathogenic species was M. tuberculosis. The sequence homology of DtxR and the endogenous mycobacterial DtxR-like repressor IdeR is approximately 60% [approaching 90% in the N-terminal half of the repressors]. In this example two, applicants have expanded this observation to demonstrate that a dominant metal ion independent repressor such a the mutant E175K DtxR is capable of attenuating virulence across wider species differences in a bacterial strain which co-regulates virulence with iron concentrations and in which there is a Fur or Fur-like repressor. (Heidrich et al., FEMS 140:253-259 (1996); Trivier et al., FEMS 141:117-127 (1996)).

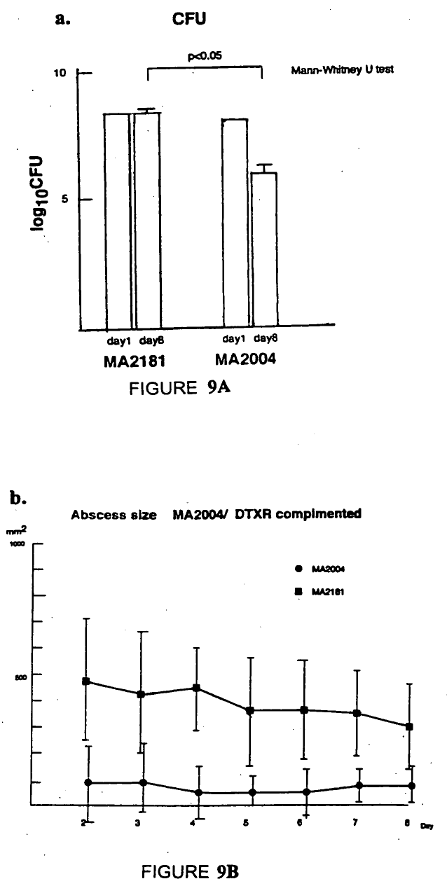

Briefly and as described above in Example 1, partial diploid analysis in reporter host strains of Escherichia coli was performed revealing dtxR(E175K) is dominant in strains which carry dtxR/dtxR(E175K). A recombinant partial diploid [dtxR(E175K)/SirR ] of Staphylococcus aureus was constructed carrying the iron-independent E175K mutant DtxR. When this strain was used in a series of mouse challenge experiments, it was found to attenuate virulence in a mouse model of staphylococcal infection See FIGS. 9A and 9B. When this strain was compared to the parent strain in a mouse skin lesion model, there was a significant decrease in CFU isolated from the lesion after 8 days associated with a significant decrease in lesion size compared to wt at each time point throughout the study. See FIGS. 9A and 9B. These data further demonstrate that activated E175K DtxR or E175K DtxR/DtxR-like (IdeR or SirR) heterodimeric repressors decrease the virulence of pathogenic microorganisms.

It is noteworthy that a dominant metal ion independent repressor from C. diphtheriae suppresses virulence in S. aureus, especially since the homology of the two repressors is at the amino acid level approaches only 30% as compared to the nearly 60% identity between IdeR from M. tuberculosis and DtxR. This means that dominant metal ion independent repressors can be employed across species barriers to selectively control gene expression to produce desired phenotypical changes.

All publications cited in the specification are indicative of the level of skill of those skilled in the art to which this invention pertains. All these publications are herein incorporated by reference to the same extent as if each individual publication were specifically and individually indicated to be incorporated by reference. In addition, U.S. Provisional Application Nos. 60/161,193, filed Oct. 22, 1999, and 60/161,292, filed Oct. 25, 1999, are hereby incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

References

1. Litwin, C. M. & Calderwood, S. B., Clin. Microbiol. Rev. 6:137-149 (1993).
2. Kochan, I., Curr. Top. Microbiol. Immunol. 60:1-30 (1973).
3. Raghu, B., Raghupati, S. & Venkatesan, P., Biochem. Mol. Biol. Int. 31:341-348 (1993).
4. Murray, M. J., Murray, A. B., Murray, M. B. & Murray, C. J., Br. Med. J. 2:1113-1115 (1978).
5. Gordeuk, V. R., McLaren, C. E., MacPhail, A. P., Deichsel, G. & Bothwell, T. H., Blood 87:3470-3476 (1996).
6. White, A., Ding, X., vanderSpek, J., Murphy, J. R. & Ringe, D., Nature 394:502-6 (1998).
7. Doukhan, L., Predich, M., Nair, G., Dussurget, O. & Mandic-Mulec, I., Gene 165:67-70 (1995).
8. Schmitt, M. P., Predich, M., Doukhan, L., Smith, I. & Holmes, R. K., Infect. Immun. 63:4284-4289 (1995).
9. Dussurget, O., Rodriguez, M. & Smith, I., Mol. Microbiol. 22:535-544 (1996).
10. Sun, L., vanderSpek, J. & Murphy, J. R., Proc. Natl. Acad. Sci. USA 95:14985-14990 (1998)
11. Valway, S. E., Sanchez, M. P. C., Shinnick, T. F., Orme, I., Agerton, T., Hoy, D., Jones, J. S., Westmoreland, H. & Onorato, I. M., N. Engl. J. Med. 338:633-639 (1998).
12. Howard, N. S., Gomez, J. E., Ko, C. & Bishai, W. R., Gene 166:181-182 (1995).
13. Zhang, Y., Lathigra, R., Garbe, T., Catty, D. & Young, D., Mol. Microbiol. 5:381-391 (1991).
14. Laboratories, B. R., in Bethesda Research Laboratory Focus, 8:9 (1986).
15. Jacobs, W. R., Jr., Kalpana, G. V., Cirillo, J. D., Pascopella, L., Snapper, S. B., Udani, R. A., Jones, W., Barletta, R. G. & B. R., B., Methods Enzymol. 204:537-555 (1991).
16. Ausubel, F. M., R., B., R. E., K., Moore, D. D., Seidman, J. G., Smith, J. A. & Struhl, K., Current Protocols in Molecular Biology (Wiley, N.Y.) (1994).
17. Tao, X., Boyd, J. & Murphy, J. R., Proc. Natl. Acad. Sci. USA (1992).
18. Miyazaki, E., Miyazaki, M., Chen, J. M., Chaisson, R. E. & Bishai, W. R., Antimicrob. Agents Chemother. 43:85-89 (1999).
19. Hendrickson, W. & Schleif, R. F., J. Mol. Biol. 178:611-628 (1984).
20. Tao, X., Zeng, H.-y. & Murphy, J. R., Proc. Natl. Acad. Sci. USA 92:6803-6807 (1995).
21. Saviola, B., Seabold, R. & Schleif, R. F., J. Mol. Biol. 278:539-548 (1998).
22. Tao, X., Schiering, N., Zeng, H.-y., Ringe, D. & Murphy, J. R., Mol. Microbiol. 14:191-197 (1994).
23. Schmitt, M. P. & Holmes, R. K., J. Bacteriol. 176:1141-1149 (1994).
24. Cole, S. T., Brosch, R., Parkhill, J., Gamier, T., Churcher, C., Harris, D., Gordon, S. V., Eiglmeier, K., Gas, S., III, C. E. B., Tekaia, F., Badcock, K., Basham, D., Brown, D., Chillingworth, T., R., C., Davies, R., Devlin, K., Feltwell, T., Gentles, S., Hamlin, N., Holroyd, S., Hornsby, T., Jagels, K., Kroghs, A., McLean, J., Moule, S., Murphy, L., Oliver, K., Osborne, J., Quail, M. A., Rajandrean, M.-A., Roger, J., Rutter, S., Seeger, K., Skelton, J., Squares, R., Squares, S., Sulston, J. E., Taylor, K., Whitehead, S. & Barell, B. G., Nature 393:537-544 (1998).
25. Husson, R. N. & Young, R. A., Proc. Natl. Acad. Sci. USA 84:1679-1683 (1987).
26. Goldberg, M. B., DiRita, V. J. & Calderwood, S. B., Infect. Immun. 58:55-60 (1990).
27. Calderwood, S. B. & Mekalanos, J. J., J. Bacteriol. 169:4759-4764 (1987).
28. Morse, S. A., Chen, C., LeFaou, A. & Mietzner, T. A., Rev. Infect. Dis. 10: s306-s310(1988).
29. Boyd, J., Oza, M. N. & Murphy, J. R., Proc. Natl. Acad. Sci. USA 87:5968-5972 (1990).
30. de Henestroza, A. R. F., Calero, S. & Barbe, J., Mol. Gen. Genet. 226:503-506 (1991).
31. Berthet, F.-X., Lagranderie, M., Gounon, P., Laurent-Winter, C., Ensergueix, D., Chavarot, P., Thouron, F., Maranghi, E., Pelicic, V., Portnoi, D., Marchal, G. & Gicquel, B., Science 282:759-762 (1998).
32. Jackson, M., Phalen, S. W., Lagranderie, M., Ensergueix, D., Chavarot, P., Marchal, G., McMurray, D. N., Gicquel, B. & Guilhot, C., Infect. Immun. 67:2867-2873 (1999).
33. Collins, F. M., Crit. Rev. Microbiol. 19:1-16 (1993).
34. Pascopella, L., Collins, F. M., Margin, J. M., Jacobs, W. R. J. & Bloom, B. R., Infect. Agents dis. 2: 282-284(1993).
35. Long, E. R. in The chemistry and chemotherapy of tuberculosis, ed. Long, E. R. (Williams & Wilkins, Baltimore), pp. 163-182 (1958).
36. Fiss, E. H., Yu, S. & Jacobs, W. R. J., Mol. Microbiol. 14:557-569 (1994).
37. Dussurget, O., Timm, J., Gomez, M., Gold, B., Yu, S., Sabol, S. Z., Holmes, R. K., Jacobs, W. R. & Smith, I., J. Bacteriol. 181:3402-3408 (1999).
38. Dussurget, O. & Smith, I., Trends Microbiol. 6:354-8 (1998).
39. Groisman, E. A. & F., H., in Two-component signal transduction, eds. Hoch, J. A. & Silhavy, T. J. (ASM Press, Washington, D.C.), pp. p319-332 (1995).
40. Dziejman, M. & Mekalanos, J. J., in Two-Component Signal Transduction, eds. Hoch, J. A. & Silhavy, T. J. (ASM Press, Washington, D.C.), pp. 305-317(1995).
41. Via, L. E., Curcic, R., Mudd, M. H., Dhandayuthapani, S., Uhner, R. J. & Deretic, V., J. Bacteriol. 178:3314-3321 (1996).
42. Buchmeier, N. A. & Heffron, F., Science 248:730-732 (1990).
43. Stojiljkovic, I., Baumler, A. J. & Heffron, F., J. Bacteriol. 177:1357-1366 (1995).
44. Lipinska, B., Fayet, O., Baird, L. & Georgopoulos, C., J. Bacteriol. 171:1574-1584 (1989).
45. Strauch, K. L. & Beckwith, J., Proc. Natl. Acad. Sci. USA 85:1576-1580 (1988).
46. Johnson, K., Charles, I., Dougan, G., Pickard, K., O'Gaora, P., Costa, G., Ali, T., Miller, I. & Hormaeche, C., Mol. Microbiol. 5:401-407 (1991).
47. Yamamoto, T., Hanawa, T., Ogata, S. & Kamiya, S., Infect. Immun. 64: 2980-2987 (1996).
48. Robertson, G. T., Elzer, P. H. & II, R. M. R., Vet. Microbiol. 49:197-207 (1996).
49. Phillips, R. W., Elzer, P. H. & II, R. M. R., Microb. Pathog. 19:227-284 (1995).
50. Chatfield, S., Strahan, K., Pickard, D., I., C., Hormaeche, C. E. & Dougan, G., Microb. Pathog. 12145-151 (1992).
51. Cameron, R. M., Stevenson, K., Inglis, N. F., Klausen, J. & Sharp, J. M., Microbiol. 140:1977-1982 (1994).
52. Gonzalez-Y-Merchand, J. A., Garcia, M. J., Gonzalez-Rico, S., Colston, M. J. & Cox, R. A., J. Bacteriol. 179:6949-6958 (1997).

1. A composition comprising a virulent or opportunistic prokaryote in which metal ion-dependent gene regulation confers a growth or an infectious advantage, said prokaryote containing a recombinant DNA molecule comprising a promoter in operable association with a sequence encoding a dominant, metal ion-independent repressor protein or a partially metal ion independent repressor protein, and a carrier.

2. The composition of claim 1 wherein said recombinant DNA molecule is contained in a non-chromosomal vector.

3. The composition of claim 1 wherein said prokaryote is a bacterium.

4. The composition of claim 3 wherein said bacterium is a member of the genus Mycobacterium.

5. The composition of claim 4 wherein said bacterium is Mycobacterium tuberculosis.

6. The composition of claim 4 wherein said bacterium is Mycobacterium leprae.

7. The composition of claim 4 wherein said bacterium is Mycobacterium avium.

8. The composition of claim 4 wherein said bacterium is Mycobacterium paratuberculosis.

9. The composition of claim 4 wherein said bacterium is Mycobacterium bovis.

10. The composition of claim 3 wherein said bacterium is a member of the genus Staphylococcus.

11. The composition of claim 10 wherein said bacterium is Staphylococcus epidermitis.

12. The composition of claim 10 wherein said bacterium is Staphylococcus aureus.

13. The composition of claim 3 wherein said bacterium is a member of the genus Streptococcus.

14. The composition of claim 13 wherein said bacterium is Streptococcus mutans.

15. The composition of claim 13 wherein said bacterium is Streptococcus pneumoniae.

16. The composition of claim 1 wherein said sequence encodes a metal ion-independent diphtheria toxin repressor (DtxR) protein.

17. The composition of claim 1 wherein said sequence encodes a metal ion-independent or a partially metal ion independent IdeR or SirR repressor protein.

18. The composition of claim 3 wherein said bacterium is a gram-positive bacterium.

19. A composition comprising a virulent or opportunistic bacterium in which metal ion-dependent gene regulation confers a growth or an infectious advantage, said bacterium containing a recombinant DNA molecule comprising a promoter in operable association with a sequence encoding a metal or metal ion-independent DtxR protein or a partially metal ion independent DtxR protein, and a carrier.

20. An isolated and purified DNA molecule consisting essentially of a sequence encoding a metal ion independent or a partially metal ion independent DtxR or homologue thereof.

21. The DNA molecule of claim 20 wherein said DtxR homolog is IdeR.

22. The DNA molecule of claim 20 wherein said DtxR homolog is SirR.

23. A recombinant DNA molecule containing a constitutive promoter element in operable association with the DNA molecule of claim 20.

24. A recombinant vector comprising a promoter element in operable association with the DNA molecule of claim 20.

25. The recombinant vector of claim 24 which is a plasmid.

26. A virulent or opportunistic prokaryote in which metal ion-dependent gene regulation confers a growth or an infectious advantage, wherein said prokaryote is transformed with a DNA molecule encoding a dominant, metal ion-independent repressor protein or a partially metal ion-independent repressor protein, and wherein said DNA molecule is expressed in said prokaryote.

27. A method of enhancing protective immunity against infection or disease caused by an opportunistic or virulent prokaryotic pathogen in which metal ion-dependent gene regulation confers a growth or an infectious advantage, comprising administering to an animal the composition of claim 1.

28. The method of claim 27 wherein said animal is a human.

29. The method of claim 27 wherein the prokaryote is in live form.

30. The method of claim 27 wherein the prokaryote is in inactivated form.

31. A method of attenuating or reducing the severity of an infection or disease caused by an opportunistic or virulent prokaryotic pathogen in which metal ion-dependent gene regulation confers a growth or an infectious advantage, comprising administering to an animal the composition of claim 1.

32. The method of claim 1 wherein said animal is a human.


SEQUENCE LISTING (36 sequences)

SEQ ID NO: 1 - DNA, 24 bp, Artificial Sequence
  Description of Artificial Sequence Primer
accagatctgccgaaaaacttcga

SEQ ID NO: 2 - DNA, 25 bp, Artificial Sequence
  Description of Artificial Sequence Primer
accagatctccgcctttagtattta

SEQ ID NO: 3 - DNA, 27 bp, Unknown Organism
  Description of Unknown Organism Native tox operator
ataattaggatagctttacctaattat

SEQ ID NO: 4 - DNA, 19 bp, Unknown Organism
  Description of Unknown Organism Illustrative polynucleotide target site
gtaggttaggctaacctat

SEQ ID NO: 5 - DNA, 25 bp, Artificial Sequence
  Description of Artificial Sequence Consensus-binding sequence
ananttaggntagnctannctnnnn

SEQ ID NO: 6 - DNA, 19 bp, Artificial Sequence
  Description of Artificial Sequence Illustrative variant polynucleotide sequence
twaggttagsctaacctwa

SEQ ID NO: 7 - protein, 230 aa, Mycobacterium tuberculosis
MNELVDTTEMYLRTIYDLEEEGVTPLRARIAERLDQSGPTVSQTVSRMERDGLLRVAGDRHLELTEKGRALAIAVMRKHRLAERLLVDVIGLPWEEVHAEACRWEHVNSEDVERRLVKVLNNPTTSPFGNPIPGLVELGVGPEPGADDANLVRLTELPAGSPVAVVVRQLTEHVQGDIDLITRLKDAGVVPNARVTVETTPGGGVTIVIPGHENVTLPHEMAHAVKVEKV

SEQ ID NO: 8 - protein, 223 aa, Cornyebacterium diptheriae
MKDLVDTTEMYLRTIYELEEEGVTPLRARIAERLEQSGPTVSQTVARMERDGLVVVASDSLQMTPTGRTLATAVMRKHRLAERLLTDIIGLDINKVHDEACRWEHVMSDEVERRLVKVKDVSRSPFGNPIPGLDELGVGNSDAAAPGTRVIDAATSMPRKVRIVQINEIFQVETDQFQLLDADIRVGSEVEIVDRDGHITLSHNGKDVELLDDLAHTIRIEEL

SEQ ID NO: 9 - protein, 174 aa, Staphylococcus epidermitis
MTVSCPPPSTSEREEQARALCLRLLTARSRTRAELAGQLAKRGYPEDIGNRVLDRLAAVGLVDDTDFAEQWVQSRRANAAKSKRALAAELHAKGVDDDVITTVLGGIDAGAERGRAEKLVRARLRREVLIDDGTDEARVSRRLVAMLARRGYGQTLACEVVIAELAAERERRRV

SEQ ID NO: 10 - protein, 225 aa, Mycobacterium leprae
MNDLVDTTEMYLRTIYDLEEEGIVTPLRARIAERPTVSQTVSRMERDGLLRVAGNRHLELTTKGRAMAIAVMRKHRLAERLLVDVIGLPWEEVHAEACRWEHVMSEDVERRLIKVLNNPTTSPFGNPIPGLLDLGAGPDASAANAKLVRLTELPSGSPVAVVVRQLTEHVDDIDLITRLKDTGVVPNARVTVETSPAGNVIIIIPGHENVTLPHEMAHAVKVEKV

SEQ ID NO: 11 - protein, 230 aa, Mycobacterium tuberculosis
MNELVDTTEMYLRTIYDLEEEGVTPLRARIAERLDQSGPTVSQTVSRMERDGLLRVAGDRHLELTEKGRALAIAVMRKHRLAERLLVDVIGLPWEEVHAEACRWEHVMSEDVERRLVKVLNNPTTSPFGNPIPGLVELGVGPEPGADDANLVRLTELPAGSPVAVVVRQLTEHVQGDIDLITRLKDAGVVPNARVTVETTPGGGVTIVIPGHENVTLPHEMAHAVKVEKV

SEQ ID NO: 12 - protein, 233 aa, Mycobacterium smegmatis
MNDLVDTTENYLRTIYDLEEEGVVPLRARIAERLDQSGPTVSQTVSRMERDGLLHVAGDRHLELTDKGRALAVAVMRKHRLAERLLVDVIGLPWEDVHAEACRWEHVMSEEVERRLVQVLENPTTSPFGNPIPGLTELAVTPGVNTEDVSLVRLTELPVGMPVAVVVRQLTEHVQGDTDLIGRLKEAGVVPNARVTVEANNNGGVMIVIPGHEQVELPHHMAHAVKVEKVEKV

SEQ ID NO: 13 - protein, 174 aa, Mycobacterium tuberculosis; identical to SEQ ID NO: 9

SEQ ID NO: 14 - protein, 228 aa, Brevibacterium lactofermentum
MKDLVDTTEMYLRTIYELEEEGIVPLRARIAERLEQSGPTVSQTVARMERDGLVHVSPDRSLEMTPEGRSLAIAVMRNDRLAERLLTDIIGLDIHKVHDEACRWEHVMSDEVERRLVEVLDDVHRSPFGNPIPGLGEIGLDQADEPDSGVRAIDLPLGENLKARIVQLNEILQVDLEQFQALTDAGVEIGTEVDIINEQGRVVITHNGSSVELIDDLAHAVRVEKVEG

SEQ ID NO: 15 - protein, 226 aa, Corynebacterium diphtheriae
MKDLVDTTEMYLRTIYELEEEGVTPLRARIAERLEQSGPTVSQTVARMERDGLVVVASDRSLQMTPTGRTLATAVMRKHRLAERLLTDIIGLDINKVHDEACRWEHVMSDEVERRLVKVLKDVSRSPFGNPIPGLDELGVGNSDAAAPGTRVIDAATSMPRKVRIVQINEIFQVETDQFTQLLDADIRVGSEVEIVDRDGHITLSHNGKDVELLDDLAHTIRIEEL

SEQ ID NO: 16 - protein, 230 aa, Mycobacterium tuberculosis; identical to SEQ ID NO: 11

SEQ ID NO: 17 - protein, 235 aa, Mycobacterium smegmatis
MNDLVDTTEMYLRTIYDLEEEGVVPLRARIAERLDQSGPTVSQTVSRMERDGLLHVAGDRHLELTDKGRALAVAVMRKHRLAERLLVDVILPWEDGVHAEACRWEHVMSEEVERRLVQVLENPTTSPFGNPIPGLTELAVTPGVNTEDVSLVRLTELPVGMPVAVVVRQLTEHVQGDTDLIGRLKEAGVVPNARVTVEANNNGGVMIVIPGHEQVELPHHMAHAVKKKVEKVEKV

SEQ ID NO: 18 - protein, 225 aa, Mycobacterium leprae; identical to SEQ ID NO: 10

SEQ ID NO: 19 - protein, 230 aa, Streptomyces lividans
MSGLIDTTEMYLRTILELEEEGVVPMRARIAERLDQSGPTVSQTVARMERDGLVSVAADRHLELTDEGRRLATRVMRKHRLAECLLVDVIGLEWEQVHAEACRWEHVMSEAVERRVLELLRHPTESPYGNPIPGLEELGETDGADPFLDEGMVSLADLDPGQEGKTVVVRRIGEPIQTDAQLMYTLRRAGVQPGSVVSVTESAGGVLVGSGGEAAELEADTASHVFVAKR

SEQ ID NO: 20 - protein, 215 aa, Staphylococcus epidermidis
MLTEEKEDYLKAILTNDGDVSFVSNKKLSQFLNIKPPSVSEMVGRLEKEGYVETKHYKGARLTEEGLKQTLDIIKRHRLLRLFLIEILQYNWEEVHQEAEILEHRISDLFVERLDKILNFPKTCPHGGVIPRGNSDAAAPGTSILNFEPGERVTVRRVRRDKTELLVYLSSKDIYIGNTVEIVSKDDTNKVIILKRNDIVTILSYENAMNIFAEK

SEQ ID NO: 21 - protein, 213 aa, Staphylococcus aureus
MLTEEKEDYLKAILTNNGDKNFVTNKILSQFLNIKPPSVSEMVGRLEKAGYVETKPYKGVRLTEDGLTHTLDIIRHRLLELFLIEILKYNWEEVHQEAEILEHRISDLFVERLDSLLNFPETCPHGGVIPRNNEYKEKYITTILNYEPGDIVTIKRVRDKTDLLIYLSSKDISIGNEVEIVSKDEMNKVIIIKRNDNVIIVSYENAMNMFAEK

SEQ ID NO: 22 - protein, 222 aa, Enterococcus faecalis
MTPNREDYLKLIFELGGDEVKVNNKQIVSGLDVSAASVSEMISKLVKEDLVEHSPYQGVQLTEKGLKKASTLIRKHRIWEVFLVEHLNYTWNDVHEEAEVLEHVTSQTLVNRLADYLNHPEFCPHGGVIPEDNQPIHEEKRQTLTDYPVGTKIRIARVLDEKELLDYLVSIDLNIQEEYTIKEIAAYEGPITIYNENKELSVSFKAANTIFVEPLIRESEEN

SEQ ID NO: 23 - protein, 215 aa, Streptococcus gordonii
MTPNKEDYLKCLYELGTRHNKITNKEIAGLMQVSPPAVTEMMKKLLAEELLIKDKKAGYLLTDLGLKLVSDLYRKHRLIEVFLVHHLGYTTEEIHEEAEVLEHTVSDHFVERLDQLLDYPKACPHGGTIPAKGELLVEKHKLTLEEAKEKGDYILARVHDNFDLLTYLERNGLQVGKTIRFLGYDDFSHLYSLEVDGQEIQLAQPIAQQIYVEKI

SEQ ID NO: 24 - protein, 217 aa, Streptococcus mutans
MTPNKEDYLKIIYELSERDEKISNKQIAEKMSVSAPAVSEMVKKLLLEDLVLKDKQAGYLLTKKGQILASSLYRKHRLIEVFLMNHLNYTADEIHEEAEVLEHTVSDVFVERLDKFLNYPKVCPHGGTIPGHGQPLVERYRTTLKGVTEMGVYLLKRVQDNFQLLKYMEQHHLKIGDELRLLEYDAFAGAYTIEKDGEQLQVTSAVASQIYIEKKAY

SEQ ID NO: 25 - protein, 216 aa, Streptococcus pneumoniae
MTPNKEDYLKCIYEIGIDLHKITNKEIAARMQVSPPAVTEMIKRMKSENLILKDKECGYLLTDLGLKLVSELYRKHRLIEVFLVHHLDYTSDQIHEEAEVLEHTVSDLFVERLDKLLGFPKTCPHGGTIPAKGELLVEINNLPLADIKEAGAYRLTRVHDSFDILHYLDKHSLHIGDQLQVKQFDGFSNTFTILSNDEDLQVNMDIAKQLYVEKIN

SEQ ID NO: 26 - protein, 216 aa, Streptococcus pyogenes; identical to SEQ ID NO: 25

SEQ ID NO: 27 - DNA, 19 bp, Artificial Sequence
  Description of Artificial Sequence Consensus sequence
gtaggttaggctaacctat

SEQ ID NO: 28 - DNA, 19 bp, Artificial Sequence
  Description of Artificial Sequence Consensus sequence
ttaggttaggctaacctaa

SEQ ID NO: 29 - DNA, 19 bp, Cornyebacterium diptheriae
ttaggatagctttacctaa

SEQ ID NO: 30 - DNA, 19 bp, Streptomyces pilosus
ttaggttaggctcacctaa

SEQ ID NO: 31 - DNA, 19 bp, Unknown Organism
  Description of Unknown Organism 16S ribosomal RNA
ccagggtatctaatcctgt

SEQ ID NO: 32 - DNA, 19 bp, Unknown Organism
  Description of Unknown Organism 19 kDa antigen
gcaggccagtgaaacctgt

SEQ ID NO: 33 - DNA, 20 bp, Unknown Organism
  Description of Unknown Organism htrA homologue
acaggtggtgctcaaccacg

SEQ ID NO: 34 - DNA, 20 bp, Unknown Organism
  Description of Unknown Organism phoP homologue
gaaggtaacgttcaaccaat

SEQ ID NO: 35 - DNA, 20 bp, Unknown Organism
  Description of Unknown Organism adhB homologue
gcaggtgaccgtcaaccgat

SEQ ID NO: 36 - DNA, 19 bp, Unknown Organism
  Description of Unknown Organism narG homologue
gaaggtcaaccaaacaaga